(19) United States
(12) Patent Application Publication
(10) Pub. No.: US 2007/0140974 A1
(43) Pub. Date: Jun. 21, 2007

(54) Targeted nanoparticles for magnetic resonance imaging

(75) Inventors: Andrew Soliz Torres, Clifton Park, NY (US); Faisal Ahmed Syud, Clifton Park, NY (US); Nichole Lea Wood, Niskayuna, NY (US); Amit Mohan Kulkarni, Clifton Park, NY (US); Mark Thomas Baillie, Atlanta, GA (US); Bahram Moasser, Schenectady, NY (US); Brian Christopher Bales, Niskayuna, NY (US); Anton Beletskii, Niskayuna, NY (US); Peter John Bonitatebus JR., Saratoga Springs, NY (US)

Correspondence Address: GENERAL ELECTRIC COMPANY;GLOBAL RESEARCH, PATENT DOCKET RM. BLDG. K1-4A59, NISKAYUNA, NY, 12309, US

(73) Assignee: General Electric Company, Schenectady, NY (US)

(21) Appl. No.: 11/304,028

(22) Filed: Dec. 15, 2005

(51) Int. Cl. A61K 49/10

(52) U.S. Cl. 424/9.323; 977/930

(57) ABSTRACT

In some embodiments, the present invention is directed to novel targeted contrast agents for magnetic resonance imaging (MRI). The present invention is also directed to methods of making such targeted MRI contrast agents, and to methods of using such MRI contrast agents. Typically, such targeted MRI contrast agents provide enhanced relaxivity, improved signal-to-noise, targeting ability, and resistance to agglomeration. Methods of making such MRI contrast agents typically afford better control over particle size, and methods of using such MRI contrast agents typically afford enhanced blood clearance rates and biodistribution.

### TECHNICAL FIELD

The present invention relates generally to nanoparticles for use in diagnostic imaging, and more specifically to nanoparticles functionalized with a targeting moiety for use as contrast agents in magnetic resonance imaging.

### BACKGROUND INFORMATION

Diagnostic imaging procedures and contrast agents are used to study organs, tissues, and diseases in a body. One example of an imaging technique comprises magnetic resonance (MR), which is a technique that uses a powerful magnetic field and radio signals to create sophisticated vertical, cross-sectional and three-dimensional images of structures and organs inside a body. Unlike conventional radiography and computed tomographic (CT) imaging, which make use of potentially harmful radiation (X-rays), magnetic resonance imaging (MRI) is based on the magnetic properties of atoms. MRI is most effective at providing images of tissues and organs that contain water, such as the brain, internal organs, glands, blood vessels and joints. When focused radio wave pulses are broadcast towards magnetically aligned hydrogen atoms in a tissue of interest, the hydrogen atoms return a signal as a result of proton relaxation. The subtle differences in the signal from various body tissues enable MRI to differentiate organs, and potentially contrast benign and malignant tissue. MRI is useful for detecting tumors, bleeding, aneurysms, lesions, blockage, infection, joint injuries, etc.

Contrast agents change the relaxation time of the tissues they occupy. Contrast agents for MRI are typically magnetic materials that enhance the relaxation time of the water protons in a close range due to a time-dependent magnetic dipolar interaction between the magnetic moments of the contrast agent and the water protons. The efficiency with which relaxation times of protons are shortened is defined as relaxivity (R1=1/T1, R2=1/T2). MRI contrast agents are either positive agents (T1 agents) that brighten the tissue that they occupy, or they are negative agents (T2 agents) that make a tissue appear darker. For in vivo diagnostics, MRI provides good resolution characteristics (ca. 2 mm), however, it offers poor sensitivity when compared with other imaging techniques. The administration of contrast agents greatly improves imaging sensitivity. Paramagnetic gadolinium (Gd) species (T1 agents) such as Gd-DTPA (e.g., OMNISCAN®) have been used clinically as contrast agents in MRI.

Superparamagnetic iron oxide nanoparticles (SPIO) have been evaluated in medicine as MRI contrast agents. Some of these products are available on the market, such as Feridex IV®, Abdoscan® and Lumirem® as contrast agents used in clinical applications for liver and spleen imaging. Superparamagnetic agents may be magnetized more than paramagnetic agents due to their ca. 1000 times higher magnetic moment, which provides a higher relaxivity (Andre E. Merbach and Eva Toth (Eds.), The Chemistry of Contrast Agents in Medicinal Magnetic Resonance Imaging, Wiley, New York, 2001, p. 38; ISBN 0471607789). Superparamagnetic iron oxide crystalline structures have the general formula [Fe₂³⁺O₃]ₓ[Fe₂³⁺O₃(M²⁺O)]₁₋ₓ where 1≧×≧0. M²⁺ may be a divalent metal ion such as iron, manganese, nickel, cobalt, magnesium, copper or a combination thereof. When the metal ion (M²⁺) is ferrous ion (Fe²⁺) and x=0, the SPIO agent is magnetite (Fe₃O₄), and when x=1, the SPIO agent is maghemite (γ-Fe₂O₃). Superparamagnetism occurs when crystal-containing regions of unpaired spins are sufficiently large that they can be regarded as thermodynamically independent, single domain particles called magnetic domains. Such a magnetic domain has a net magnetic dipole that is larger than the sum of its individual unpaired electrons. In the absence of an applied magnetic field, all the magnetic domains are randomly oriented with no net magnetization. An external magnetic field causes the dipole moments of all magnetic domains to reorient, resulting in a net magnetic moment. T1, T2 and T2* relaxation processes are shortened by SPIO. At room temperature and at 1.5 Tesla magnetic field, the R2 relaxivity is in the range 40-60 mM⁻¹s⁻¹ and R1 relaxivity is in the range 10-20 mM⁻¹s⁻¹. The relaxivities are substantially larger than that of paramagnetic agents such as Gd-DTPA, for which R2 is 4 mM⁻¹s⁻¹ and R1 is 3 mM⁻¹s⁻¹. The relaxivities of SPIO are dependent on a variety of factors, such as particle size, composition, coating chemistry, surface charge and particle stability. The ratio of relaxivities, R2/R1, is commonly used to quantify the type of contrast produced by SPIO, At R2/R1 values less than 10, the T1 (positive) effect of SPIO can be emphasized using T1-weighted sequences. At R2/R1 values greater than 10, the T2 effect dominates and the agent is a T2/T2* agent. A positive contrast technique has been recently used to visualize cells labeled with SPIO (Mag. Res. Medicine 2005:53: 999-1005, C. H. Cunningham et al). SPIO agents thus offer enormous versatility in their use as a positive or negative agent.

Contrast agent specificity is a desired property for enhancing signal-to-noise ratio at a site of interest and providing functional information through imaging. Natural biodistribution of contrast agents depends upon the size, charge, surface chemistry and administration route. Contrast agents may concentrate at healthy tissue or lesion sites and increase the contrast between the normal tissue and the lesion. In order to increase contrast, it is necessary to concentrate the agents at the site of interest and increase relaxivity. In addition, it is also desirable to increase the uptake of the agents by diseased cells in relation to healthy cells.

Most contrast agents are somewhat organ-specific due to the fact that they are excreted either by the liver or by the kidneys. Initial studies using gadolinium chelates as receptor-directed agents required a high level of contrast agent for a significantly reduced relaxation (Eur. Radiol. 2001. 11:2319-2331, Y.-X. J. Wang, S. M. Hussain, G. P. Krestin). Compared to the gadolinium chelates, magnetite particles possess about two to three orders of magnitude greater magnetic susceptibility (Eur. Radiol. 2001. 11:2319-2331, Y.-X. J. Wang, S. M. Hussain, G. P. Krestin). Therefore, iron oxide contrast agents potentially offer a stronger signal at a lower dosage than gadolinium chelates. The higher sensitivity of iron oxide agents provides additional benefits due to the limited number of targets available to bind with in a given tissue.

There are a variety of magnetic nanoparticles such as magnetodedrimers, magnetoliposomes and polymer-coated nanoparticles (such as dextran, polyvinyl alcohol, etc.) that are made up of crystalline superparamagnetic iron oxide nanoparticles embedded in an organic coating. These nanoparticles are generally evaluated for magnetic separation, cell tracking and imaging. Some are currently being tested for clinical applications, such as liver and spleen imaging, bowel contrast and MR angiography. The hydrodynamic diameters (D_{H}) of these agents are generally in the range of about 20 nm to about 400 nm, and most of these agents clear from the blood rapidly by the uptake of the reticuloendothelial system (RES). They are primarily contrast agents for the organs constituting the RES system, specifically the liver. Smaller particle sizes are generally necessary in order to image other organs.

Most of the commercial contrast agents (D_{H}=80-150 nm), and those that are in phase 3 trials (D_{H}=20-80 nm), are based on dextran or dextran derivatives, where relatively small size particles are employed. However, dextran coatings have been claimed to be unstable at the alkaline conditions of the particle synthesis, and their chemical composition has therefore been questioned. Additionally, dextran-induced anaphylactic reactions present potential problems (R. Weissleder U.S. Pat. No. 5,492,814).

Conventionally, iron oxide nanoparticles are synthesized and precipitated from alkaline aqueous solutions in the presence of water soluble organic molecules such as dextran, and such nanoparticles generally have an organic coating. Nanoparticles obtained by such methods tend to have a broad size distribution of the superparamagnetic iron oxide, and, as a result, the coated particles also exhibit a broad size distribution. In addition, this method provides little control over the degree of coating leading to particles containing multiple iron oxide nanoparticles within a single agent. Extensive manufacturing techniques, including multiple purification and size separation steps, are necessary to obtain the desired particle sizes. Particle size, as well as the organic coating composition, is very important as it directly affects the pharmacokinetics of the nanoparticles. The size of the iron oxide directly relates to the superparamagnetism and the relaxivity of the agent. Therefore, a broad size distribution generally translates into an average sensitivity.

Nanoparticles obtained using conventional methods also have a low level of crystallinity, which significantly impacts the sensitivity of the contrast agent. Moreover, nanoparticles tend to agglomerate due to their high surface energy, which is a significant problem encountered during synthesis and purification steps. Such agglomeration increases the size of the particle, resulting in rapid blood clearance as well as reducing targeting efficiency, and may result in a reduction in relaxivity. Size, blood circulation time and the organic coating affect the targeting efficiency in different ways. When large particles are employed, only a few targeting ligands may be attached before the particles become large enough to activate the RES, resulting in almost instantaneous clearance from the blood and failure of the agent to reach the intended target. Smaller particle sizes may be much “stickier” at the sites where the recognition between the biomarker and the ligand occurs. When coatings are globular, reactive sites intended for ligand attachment are generally hindered, thereby reducing conjugation efficiency. In addition, once bound, ligands may reside in the interior of globular coatings, preventing easy access to the biomarkers.

Current imaging agents and modalities primarily provide anatomical information. However, underlying disease states are biochemical processes that propagate the disease well before outward physical symptoms appear. Having the ability to image the biochemical pathways, or specific markers in the pathways (biomarkers or physiological changes), in the early stages of the disease would provide functional information. This may be termed “targeted molecular imaging.” To illustrate, in the case of atherosclerosis, fatty streaks or lesions form due to a cascade of chemical events long before plaque formation. Furthermore, the body is able to adapt to this by increasing the outer diameter of the vasculature wall so that any accumulating plaque is masked. The plaque only becomes detectable once it reaches a critical size, resulting in blocked blood flow, or when it ruptures, which may lead to thrombus (blood clot) formation, resulting in acute myocardial infarction or death.

Contrast agents that are targeted towards particular molecular markers that are able to detect the increased presence of the crucial chemical biomarkers, and thereby provide biochemical information on the early presence of a specific disease state, are needed. Molecular contrast agents capable of targeting sites of active inflammation and responding to the physiological signature of a lesion are needed to address the medical need for early diagnosis and treatment of disease. One of the major developmental needs in molecular imaging and targeted delivery of contrast agents is the identification of the biomarkers. Contrast agents, however, have inherent problems that limit targeting efficiency, such as low sensitivity, low signal-to-noise ratio, large particle sizes, rapid blood clearance, low efficiency of ligand attachment and the accessibility of ligands to the biomarkers' targets.

Previous examples of targeted delivery of contrast agents involved using iron oxide nanoparticles coated with cross-linked dextran and subsequently adding antibodies or peptides (Kelly, K. A., Allport, J. R., Tsourkas, A., Shinde-Patil, V. R., Josephson, L., and Weissleder, R. (2005) Circ Res 96, 327-336; Wunderbaldinger, P., Josephson, L., and Weissleder, R. (2002) Bioconjug Chem 13, 264-268). While conjugation of the molecules and delivery of agent to a site of interest was accomplished, the agents became very large (>65 nm) upon bioconjugation and demonstrated very low blood half-life (<50 minutes) which could dramatically affect efficacy in humans. Another example includes ionic functionalization of monodisperse 9 nm iron oxide cores with 2,3-dimercaptosuccinic acid (DMSA) and conjugating maleimide-functionalized Her2-specific antibodies to the DMSA-nanoparticles (Huh, Y. M., Jun, Y. W., Song, H. T., Kim, S., Choi, J. S., Lee, J. H., Yoon, S., Kim, K. S., Shin, J. S., Suh, J. S., and Cheon, J. (2005) J Am Chem Soc 127, 12387-12391; Jun, Y. W., Huh, Y. M., Choi, J. S., Lee, J. H., Song, H. T., Kim, S., Yoon, S., Kim, K. S., Shin, J. S., Suh, J. S., and Cheon, J. (2005) J Am Chem Soc 127, 5732-5733). The resulting non-covalently bioconjugated nanoparticles have a hydrated diameter of 28 nm and demonstrated targeting to cancer cells in vivo. The primary limitation of this technology is the measured Mₛₐₜ values of these agents is between 43-60 emu/g for 4-6 nm core nanoparticles. These relatively low Mₛₐₜ values would have profound implications for imaging of these particles when they localize at the disease site of interest. Additionally, the DMSA-nanoparticle interaction is ionic and not covalent which could reduce the ability of the targeting molecule to remain attached to the nanoparticle following injection. In summary, it would be of significant value to identify novel strategies to covalently attach targeting molecules to highly magnetic (>60 emu/g), monodisperse nanoparticles with cores of less than 10 nm diameter.

There exists a tremendous need for advancing the limits of detection, increasing resolution, providing full-body images, obtaining information at a molecular level, detecting diseases in their early stages, and obtaining physiological information through MRI investigation. Such challenges require an improvement in contrast agent sensitivity, selectivity, blood-circulation time and also characterization of biomarkers and targeting ligands.

As a result of the foregoing, a method and/or composition by/with which nanoparticles would provide enhanced relaxivity, signal-to-noise ratio and targeting abilities with resistance to agglomeration and an ability to control particle size, blood clearance rate and biodistribution would be extremely useful.

### BRIEF DESCRIPTION OF THE INVENTION

In some embodiments, the present invention is directed to novel targeted contrast agents for magnetic resonance imaging (MRI). The present invention is also directed to methods of making such targeted MRI contrast agents, and to methods of using such MRI contrast agents. Typically, such targeted MRI contrast agents provide enhanced relaxivity, improved signal-to-noise, targeting ability, and resistance to agglomeration. Methods of making such MRI contrast agents typically afford better control over particle size, and methods of using such MRI contrast agents typically afford enhanced blood clearance rates and biodistribution.

In some embodiments, the present invention is directed to targeted MRI contrast agents comprising: (a) an inorganic-based magnetic core; (b) an organic-based non-magnetic coating disposed about and bonded to the inorganic-based magnetic core such that, in the aggregate, the magnetic core and the non-magnetic coating provide for a core/shell nanoparticle; and (c) a targeting species attached to the core/shell nanoparticle such that, in the aggregate, the core/shell nanoparticle and the targeting species provide for a targeted MRI contrast agent.

In some embodiments, the present invention is directed to a method of making such above-described targeted MRI contrast agents, the method comprising the steps of: a) synthesizing a core of a nanoparticle; b) synthesizing a shell of the nanoparticle so that the core of the nanoparticle is substantially covered by the shell; and c) attaching a targeting molecule to the shell of the nanoparticle.

In some embodiments, the present invention is directed to methods of using the above-described targeted contrast agent in an imaging technique such as MRI. Such uses can involve delivery to cells in vitro and/or delivery to a mammalian subject in vivo.

The foregoing has outlined rather broadly the features of the present invention in order that the detailed description of the invention that follows may be better understood. Additional features and advantages of the invention will be described hereinafter which form the subject of the claims of the invention.

### BRIEF DESCRIPTION OF THE DRAWINGS

For a more complete understanding of the present invention, and the advantages thereof, reference is now made to the following descriptions taken in conjunction with the accompanying drawings, in which:

FIG. 1 depicts an idealized cross-sectional view of a core/shell nanoparticle as utilized in targeted MRI contrast agents, in accordance with some embodiments of the present invention;

FIG. 2 depicts an idealized cross-sectional view of a targeted MRI contrast agent, in accordance with some embodiments of the present invention;

FIG. 3 depicts, in flow diagram form, a method of making a targeted MRI contrast agent, in accordance with some embodiments of the present invention;

FIG. 4 depicts a synthetic route for attaching targeting moieties to nanoparticles, in accordance with some embodiments of the present invention;

FIG. 5 depicts polyethylene imine (PEI) coated nanoparticles having numerous available secondary amines for coupling to N-acetylated peptides, in accordance with some embodiments of the present invention;

FIG. 6 is a micrograph of MRI contrast agents comprising NHS ester-Cypher5E dye covalently bound to the PEI-coated nanoparticles and delivered to phagocytic cells stained with Cell Tracker Green dye, in accordance with some embodiments of the present invention; and

FIG. 7 is a micrograph of RKO cells after incubation with fluorescein tagged AESTYHHLSLGYMYTLN-NH2, in accordance with some embodiments of the present invention.

### DETAILED DESCRIPTION OF THE INVENTION

In some embodiments, the present invention is directed to novel targeted contrast agents for magnetic resonance imaging (MRI). The present invention is also directed to methods of making such targeted MRI contrast agents, and to methods of using such MRI contrast agents. Typically, such targeted MRI contrast agents provide enhanced relaxivity, improved signal-to-noise, targeting ability, and resistance to agglomeration. Methods of making such MRI contrast agents typically afford better control over particle size, and methods of using such MRI contrast agents typically afford enhanced blood clearance rates and biodistribution.

1. Targeted Core/Shell Nanoparticle-Based MRI Contrast Agents

Generally, the targeted MRI contrast agents described herein are core/shell nanoparticle-based. Accordingly, in some embodiments, the present invention is directed to targeted MRI contrast agents comprising: (a) an inorganic-based magnetic core; (b) an organic-based non-magnetic coating disposed about and bonded to the inorganic-based magnetic core such that, in the aggregate, the magnetic core and the non-magnetic coating provide for a core/shell nanoparticle; and (c) a targeting species attached to the core/shell nanoparticle such that, in the aggregate, the core/shell nanoparticle and the targeting species provide for a targeted MRI contrast agent.

In some embodiments directed to a targeted MRI agent, the above-mentioned inorganic-based magnetic core comprises a material selected from the group consisting of transition metals, alloys, metal oxides, metal nitrides, metal carbides, metal borides, and combinations thereof. In some such embodiments, the inorganic-based magnetic core comprises a material that is superparamagnetic. In some such embodiments, the inorganic-based magnetic core comprises iron oxide. While the material of which such inorganic-based material is comprised is not particularly limited, such magnetic cores must generally comprise a material suitable for enhancing MRI when employed as a contrast agent. Such inorganic-based magnetic cores are generally nanoparticles and generally comprise a diameter of less than about 100 nm, typically less than about 50 nm, and more typically less than about 30 nm. As used herein, the term “inorganic-based” refers to material that is predominately not hydrocarbon. Generally, this precludes polymeric material.

In some embodiments directed to a targeted MRI agent, the above-mentioned organic-based non-magnetic coating comprises a polymer coating. In some such embodiments, the polymer coating comprises silane modified polyethylene imine (PEI). In some or other embodiments directed to a targeted MRI agent, the above-mentioned organic-based non-magnetic coating comprises a non-polymer coating. In some such latter embodiments, the non-polymer coating is aminopropyl silane. Generally, these coatings are functional in that they permit the attachment of targeting species either directly or via linker species. Note that, as used herein, the term “organic-based” is used to describe hydrocarbon-based species, wherein such hydrocarbons can be substituted to further include one or more functional moieties (e.g., halogens, amino groups, silane groups, etc.). In some embodiments, such organic-based non-magnetic coatings are selected such that they permit multiple ligand conjugation and/or do not increase the diameter of the resulting core/shell nanoparticle much beyond the diameter of the inorganic-based magnetic core. In some or other embodiments, the organic-based non-magnetic coatings provide stability to the nanoparticle cores and can permit the incorporation of therapeutic agents.

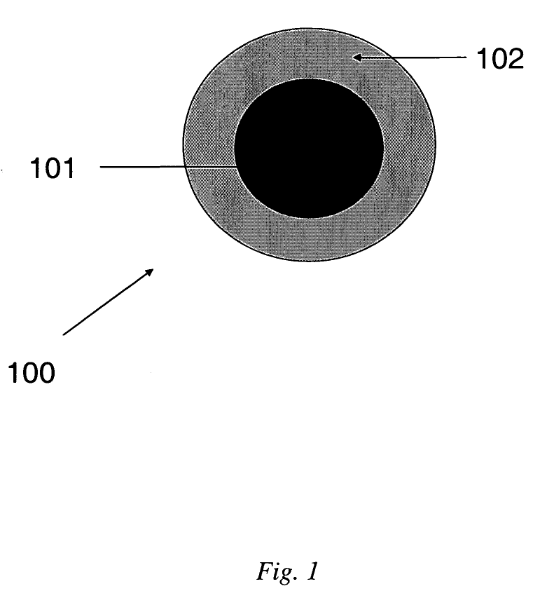

As described above, the targeted MRI contrast agent comprises a core/shell nanoparticle. Referring to FIG. 1, an idealized core/shell nanoparticle 100 is depicted comprising a core 101 and a shell 102. Such core/shell nanoparticles typically have a composite diameter of less than about 100 nm. It will be understood by those of skill in the art that such spherical representations are idealized, and that such core/shell nanoparticles are generally of an irregular shape. In some such embodiments, such core/shell nanoparticles are monodisperse. Additionally, in some embodiments, the shell can be seen as comprising multiple subshells, i.e., a multi-layered shell. Exemplary such core/shell nanoparticles are described in Bonitatebus et al., U.S. Pat. No. 6,797,380 and Bonitatebus et al., U.S. patent application Ser. No. 10/208,945.

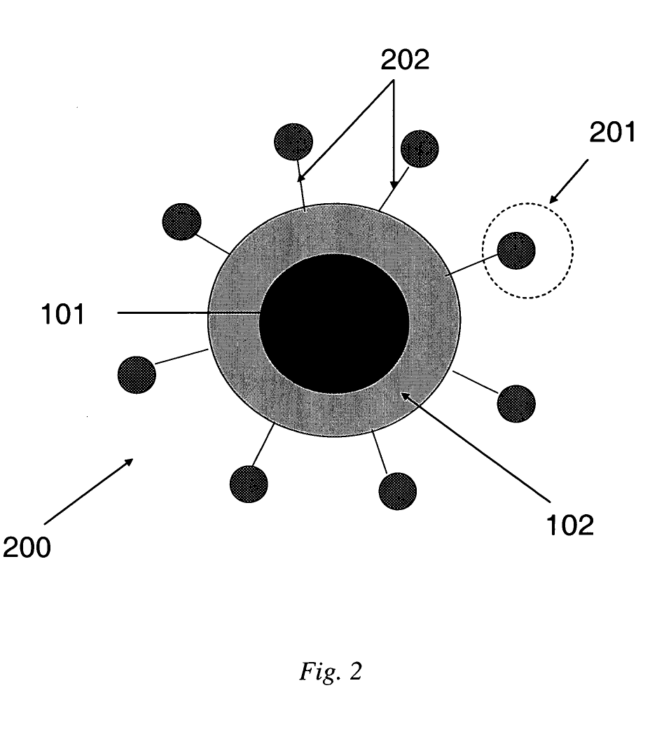

As described above, in addition to a core/shell nanoparticle, the targeted MRI contrast agents further comprise a targeting species, wherein the targeting species is attached to the core/shell nanoparticle. Typically, such attachment involves a covalent linkage (although non-covalent attachment is also permissible), and an exemplary embodiment of such a targeted MRI contrast agent is depicted in FIG. 2. Referring now to FIG. 2, such a targeted MRI contrast agent 200 comprises the core/shell nanoparticle 100 depicted in FIG. 1, and targeting species 201 attached to the shell 102 of the core/shell nanoparticle 100 via a linker species 202.

Generally, targeting species are ligands or other moieties that direct the MRI contrast agent to a specific organ or disease site. In some embodiments, the targeting molecule is a peptide. Suitable peptides include, but are not limited to, AEPVYQYELDSYLRSYY (SEQ ID NO: 1), AEFFKLGPNGYVYLHSA (SEQ ID NO: 2), AELDLSTFYDIQYLLRT (SEQ ID NO: 3), AESTYHHLSLGYMYTLN (SEQ ID NO: 4), and combinations thereof. In some or other embodiments, the targeting molecule is selected from the group consisting of a protein, an oligonucleotide; a small organic molecule, a peptide nucleic acid, and combinations thereof.

In some embodiments, targeting species are attached to the core/shell nanoparticle via a linker species such as 1-ethyl-3-(3-Dimethylaminopropyl) carbodiimide Hydrocholoride (EDC). The linker can include any linking moiety that attaches the targeting species to the nanoparticle through a first moiety. The linker can be as short as one carbon or a long polymeric species such as polyethylene glycol, polylysine or other polymeric species normally used in the pharmaceutical industry for modulating pharmacokinetic and biodistribution characteristics of such agents. Other linkers of varying length include Cᵢ-C₂₅₀ length with one or more heteroatoms selected from oxygen, sulfur, nitrogen, and phosphorus, and optionally substituted with halogen atoms. In a particular embodiment, the linker comprises at least one of an oligomeric or polymeric species made of natural or synthetic monomers, oligomeric or polymeric moiety selected from a pharmacologically acceptable oligomer or polymer composition, an oligo- or poly-amino acid, peptide, saccharide, a nucleotide, and an organic moiety with 1-250 carbon atoms, either individually or in combination thereof. The organic moiety with 1-250 carbon atoms may contain one or more heteroatoms such as oxygen, sulfur, nitrogen, and phosphorus and be optionally substituted with halogen atoms at one or more places.

The first moiety may be an extension of the linker, formed by the reaction of a reactive species on the linker with a reactive group on the nanoparticle. Examples of reactive species and the reactive group include, but are not limited to, activated esters (such as N-hydroxysuccinimide ester, pentafluorophenyl ester), a carbodiimide, a phosphoramidite, an isocyanate, an isothiocyanate, an aldehyde, an acid chloride, a sulfonyl chloride, a maleimide, an alkyl halide, an amine, a phosphine, a phosphate, an alcohol, a carboxylic acid, or a thiol with the proviso that the reactive species and reactive group are matched to undergo a reaction yielding covalently linked conjugates.

2. Methods of Making Core/Shell Nanoparticle-Based Targeted MRI Contrast Agents

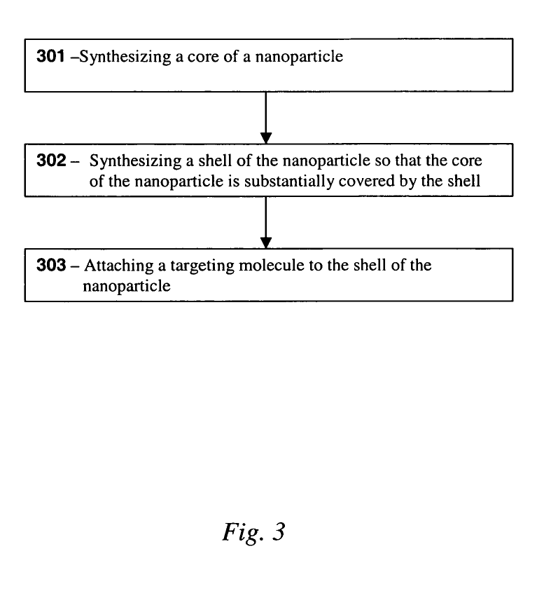

In some embodiments, methods of making the above-described targeted MRI contrast agents comprise the steps of: a) synthesizing a core of a nanoparticle 301; b) synthesizing a shell of the nanoparticle so that the core of the nanoparticle is substantially covered by the shell 302; and c) attaching a targeting molecule to the shell of the nanoparticle 303 as depicted in FIG. 3.

In some embodiments, the inorganic-based magnetic core has improved magnetization through improved crystallinity. This improved crystallinity is largely a function of how the core is made. Control over the core's size is accomplished, e.g., through control over metal oxide core size and size distribution, and through control over shell thickness by using preformed polymers of known length. Magnetic metal oxide cores, for example, can be stabilized and prevented from agglomerating by the oligomerization/polymerization of a stabilizing surfactant shell and covalent attachment of the polymer chains to the stabilizing surfactant shell. Such coating chemistry allows for control over polarity, charge, responsive nature and flexibility in the design of particles for specific sites and purposes.

3. Methods of Using Core/Shell Nanoparticle-Based Targeted MRI Contrast Agents

In some embodiments, the present invention is directed to methods of using the above-described targeted MRI contrast agent. In some such embodiments, the contrast agent is delivered to a cell in vitro, and such delivery of the contrast agent to the cell can be monitored. In some such embodiments, the contrast agent is delivered to a subject in vivo, and such delivery of the contrast agent to the subject can likewise be monitored. In some such latter embodiments, monitoring delivery of the contrast agent is accomplished via an imaging technique including but not limited to MRI, optical imaging (including optical coherence tomography), computer tomography, positron emission tomography, and combinations thereof.

Targeted MRI contrast agents can be receptor-directed by utilizing bio-recognition processes in order to concentrate such contrast agents at a target locus, thus amplifying the signal at the target locus and enhancing the image of the area. In some embodiments, this allows for specific targeting of novel MRI contrast agents to sites of disease related to up-regulation of the urokinase receptor (uPAR) or other disease biomarkers for the purpose of diagnostic molecular imaging or therapeutics. Disease biomarkers include, but are not limited to, peptides, proteins, small molecules and nucleic acids. Attachment of the peptides (i.e., targeting species) specific for uPAR to core/shell nanoparticles allows for targeting of the MRI contrast agents to sites of disease characterized by areas of up-regulation of uPAR. The nanoparticle attached peptides specific for uPAR are also be capable of inhibiting the binding of uPA:uPAR to vitronection or integrin. Specifically, peptide AESTYHHLSLGYMYTLN (SEQ ID NO: 4) is capable of binding uPAR and inhibiting binding of integrin (U.S. Pat. No. 6,794,358). Peptides AEPVYQYELDSYLRSYY (SEQ ID NO: 1), AEFFKLGPNGYVYLHSA (SEQ ID NO: 2), AELDLSTFYDIQYLLRT (SEQ ID NO: 3) are capable of binding uPAR and inhibiting binding of vitronectin (U.S. Pat. No. 6,794,358). Additionally, urokinase-type plasminogen activator and the urokinase-type plasminogen activator receptor convert plasminogen into plasmin which is responsible for localized cell surface proteolytic activity (Ellis et al, J. Biol. Chem., 264:2185-2188 (1989)). This occurs during migration of normal and tumor cells.

The MRI contrast agents can be monitored for uptake via imaging for diagnosis of several diseases including, but not limited to, cancer and inflammatory diseases such as rheumatoid arthritis (RA), chronic obstructive pulmonary disease (COPD) and multiple sclerosis (MS).

The methods of making targeted MRI contrast agents, as described herein, provide for core/shell nanoparticle-based targeted MRI contrast agents comprising any combination of the following: non-aggregated structures, non-aggregated crystals, uniform and enhanced magnetic properties per particle, longer blood half-life and access through small openings for the imaging of organs and tissues that are not a part of the reticuloendothelial system (RES), the option of being used as blood pool agents or site-specific contrast agents, a larger effective volume for water diffusion as well as a closer proximity of the water molecules to a superparamagnetic oxide (SPMO) core that enhances signal intensity and contrast, enhanced targeting ability and the detection of early stages of disease.

The following examples are included to demonstrate particular embodiments of the present invention. It should be appreciated by those of skill in the art that the methods disclosed in the examples that follow merely represent exemplary embodiments of the present invention. However, those of skill in the art should, in light of the present disclosure, appreciate that many changes can be made in the specific embodiments described and still obtain a like or similar result without departing from the spirit and scope of the present invention.

### EXAMPLE 1

This Example illustrates the synthesis and characterization of SPIO nanoparticles and preparation of PEI-silane coated SPIO nanoparticles.

Synthesis of 5 nm SPIO nanoparticles. A 25 mL, 3-neck Schlenk flask was fitted with a condenser, stacked on top of a 130 mm Vigreux column, and a thermocouple. The condenser was fitted with a nitrogen inlet and nitrogen flowed through the system. The Schlenk flask and Vigreux column were insulated with glass wool. Trimethylamine-N-oxide (Aldrich, 0.570 g, 7.6 mmol) and oleic acid (Aldrich: 99+%, 0.565 g, 2.0 mmol) were dispersed in 10 mL of dioctylether (Aldrich: 99%). The dispersion was heated to 80° C. at a rate of about 20° C./minutes. Once the mixture had reached ˜80° C., 265 μL of Fe(CO)₅ (Aldrich: 99.999%, 2.0 mmol) was rapidly injected into the stirring solution through the Schlenk joint. The solution turned black instantaneously, with a violent production of a white “cloud.” The solution rapidly heated to ˜-120-140° C. Within 6-8 minutes the reaction pot cooled to 100° C. at which it was kept and stirred for 75 minutes. After stirring at ˜-100° C. for 75 minutes, the temperature was increased to ˜-280° C. at a rate of about 20° C./min. After the solution stirred for 75 minutes, the heating mantel and glass wool were removed to allow the reaction to return to room temperature. Once at room temperature, an aliquot was removed and dissolved in toluene for size measurement using dynamic light scattering (DLS), image analysis using transmission electronic microscopy (TEM), and elemental analysis using energy dispersive x-ray analysis (EDX).

To prepare a sample for vibrating sample magnetometer analysis and elemental analysis approximately 5-10 mL of crude reaction solution was added to 20 mL of isopropanol, and the solution was centrifuged for 10 minutes at 3000 rpm. The supernatant was decanted, an additional 20 mL of isopropanol was added, and again the precipitate was collected by centrifugation. The precipitated iron oxide nanoparticles were allowed to air-dry overnight, yielding a black magnetic powder.

Saturation Magnetization. The saturation magnetization (Mₛₐₜ) of the precipitated SPIO nanoparticles was measured using a vibrating sample magnetometer (VSM). Elemental analysis was performed on the magnetic powder to determine the concentration of Fe, and the Mₛₐₜ was calculated in units of emu/g Fe for each sample. The Mₛₐₜ for bulk γ-Fe₂O₃ and Fe₃O₄ is known to be ˜104 emu/g Fe and ˜127 emu/g Fe, respectively. Although some reactions yielded SPIO agents with Mₛₐₜ values lower than 100 emu/g Fe, Mₛₐₜ values for the disclosed SPIO agents typically ranges from about 100 emu/g Fe to about 120 emu/g Fe (Table 1).

TABLE 1
Saturation Magnetization, Mₛₐₜ

| Mean Size (nm) | Mₛₐₜ (emu/g Fe) |
|---|---|
| 4.80 | 116.60 |
| 5.46 | 124.30 |
| 4.58 | 83.60 |
| 5.00 | 123.20 |
| 4.60 | 84.68 |
| 3.95 | 101.57 |
| 4.92 | 97.40 |
| 4.25 | 99.01 |

Preparation of PEI-silane coated SPIO nanoparticles. To a vial containing 3.25 mg Fe/mL 5 nm SPIO in tetrahydrofuran (4.0 mL, 13 mg Fe, 0.232 mmol) was added tetrahydrofuran (10 mL) followed by 50% PEI silane in isopropyl alcohol (2.0 mL) and the resulting cloudy solution was sonicated for 2 hours. Isopropanol (4.0 mL) was then added and the solution was sonicated for an additional 16 hours. Concentrated NH₄OH (1.0 mL, 14.8 mmol) was then added and the solution was stirred at room temperature for 4 hours. The solution was then diluted with H₂O (10 mL) and extracted with hexanes (3×10 mL) and etoleic acid (3×10 mL). Any remaining organics in the aqueous layer were removed in vacuo. The resulting homogeneous aqueous solution was passed through a 200 nm followed by a 100 nm syringe filter. The solution was then diluted with H₂O (10 mL total volume) and purified using a 100 kDa MW cutoff filter (2680× g until ˜3 mL of solution remained). The centrifuge filtration process was carried out a total of 6 times. The final pH of the solution was adjusted to about 7.4 to about 7.7 using concentrated HCl as necessary.

### EXAMPLE 2

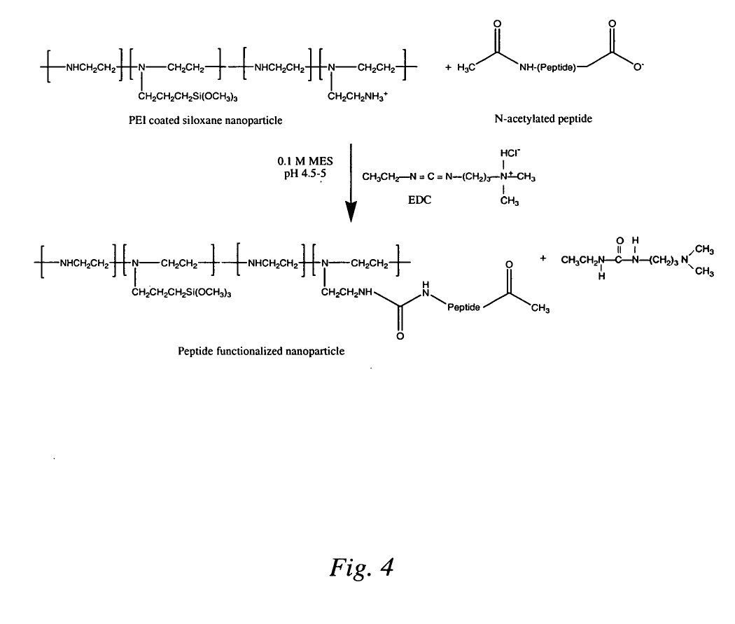

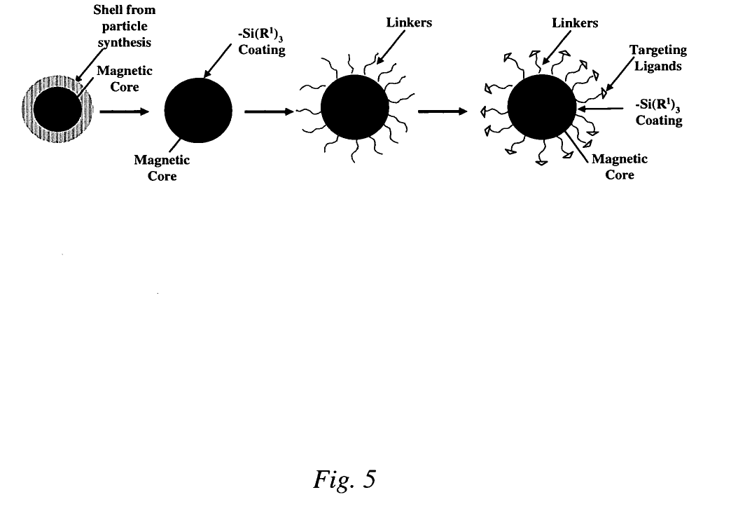

This Example illustrates attachment of peptides to PEI-coated siloxane core/shell nanoparticles. Polyethylene imine-coated siloxane core/shell naonoparticles are conjugated to N-acelyated peptides utilizing EDC. The reaction takes place in 0.1M MES, pH 4.5-5, as depicted in the synthetic scheme of FIG. 4. The polyethylene imine (PEI)-coated core/shell nanoparticles have numerous available secondary amines for coupling to N-acetylated peptides with the amount of conjugation controlled to achieve maximum binding efficiency to the biological target, as depicted in FIG. 5.

### EXAMPLE 3

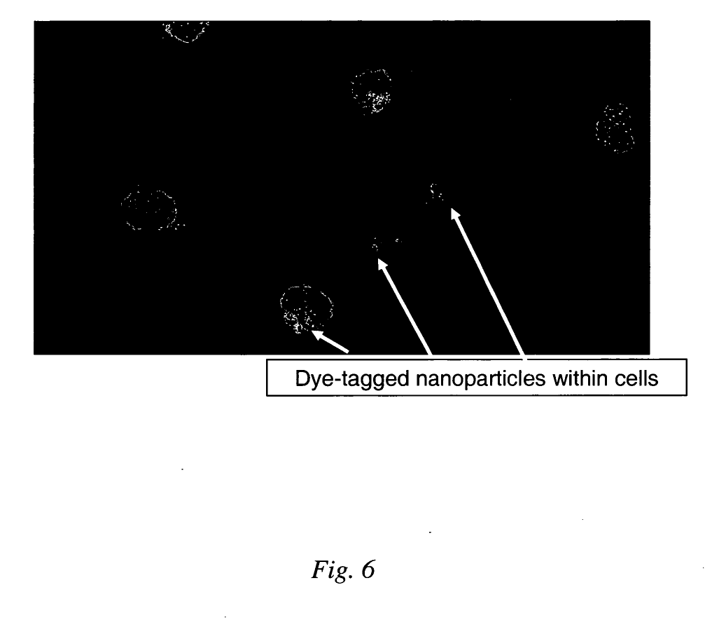

This Example is illustrative of cell uptake studies. NHS ester-Cypher5E dye was covalently bound to the PEI-coated nanoparticles. These amine-coupled dyes indicate the uptake of these nanoparticles into phagocytic cells and demonstrate the utility of the free amines of the PEI coating for attachment using NHS ester chemistry (similar to coupling chemistry for peptide, etc). Peptides can be coupled to these particles in a similar manner for uptake in non-phagocytic disease-specific cells expressing biomarkers of interest for diagnosis. FIG. 6 is a micrograph of MRI contrast agents comprising NHS ester-Cypher5E dye covalently bound to the PEI-coated nanoparticles and delivered to phagocytic cells stained with Cell Tracker Green dye, in accordance with some embodiments of the present invention.

Peptide-functionalized cationic nanoparticles could also deliver oligonucleotides to disease-specific sites for therapeutic or diagnostic purposes.

### EXAMPLE 4

This Example illustrates the design and synthesis of peptides for targeting uPAR. Peptides that bind uPAR may be derived from a variety of sources including peptide fragments of proteins that bind uPAR or combinatorial libraries such as Phage Display. The binding may also potentially inhibit the activity of uPAR, and hence be an inhibitor. An example of such a peptide is an integrin fragment AEPVYQYELDSYLRSYY-NH2 (WO 97/35969). As with standard peptide chemistry, the sequence above may be synthesized using solid-phase peptide synthesis, incorporating a label at the N-terminus. The label could be attached to the alanine, A, in the above sequence.

Peptides were synthesized using standard solid phase techniques with N⁶⁰-Fmoc-protected amino acids using 2,4-dimethoxybenzhydrylamine resin (Rink Amide AM) on a 25 μmole scale (Fmoc=fluorenylmethoxycarbonyl). The peptides were synthesized using a Rainin/Protein Technology Symphony solid phase peptide synthesizer (Woburn, Mass.). Prior to any chemistry, the resin was swelled for one hour in methylene chloride, and subsequently exchanged out with DMF (dimethylformamide) over half-hour or more. Each coupling reaction was carried out at room temperature in DMF with five equivalents of amino acid. Reaction times were typically 45 minutes with reaction times of 1 hour for residues that were expected to be difficult to couple (for example, coupling isoleucine, I, to proline, P, in the IPP sequence). The coupling reagent used was HBTU (O-Benzotriazolyl-1-yl-N,N,N′,N′-tetramethyluronium hexafluorophosphate), with NMM (N-methylmorpholine) as the base. For each step the coupling agent was delivered at a scale of five equivalents relative to the estimated resin capacity, and the reaction was carried out in 2.5 mL of 0.4 M NMM solution in DMF. The reactions did not perturb the side-chains of the amino acids, which were typically protected with acid labile groups if reactive groups were present. Generally, the tyrosine, threonine and serine side chains were protected as the corresponding tert-butyl ethers. The glutamic acid side chain was protected as the corresponding tert-butyl ester. The lysine and omithine side chains were Boc protected. The glutamine side chain was protected as the γ-triphenylmethyl derivative, and the arginine side chain was protected as the 2,2,5,7,8-Pentamethyl-chromane-6-sulfonyl derivative.

Following each coupling reaction, the N-terminal Fmoc-protected amine was deprotected by applying 20% piperidine in DMF twice at room temperature for approximately 15 minutes. After the addition of the last residue, the resin, still on the peptide synthesizer, was rinsed thoroughly with DMF and methylene chloride.

To couple a fluorescein dye such as 5(6)-carboxyfluorescein to the N-terminus of the peptide, the dye, HBTU and NMM were added to the resin in the same manner as the amino acids. After the reaction, the resin was thoroughly washed with DMF and methylene chloride and dried under a stream of nitrogen. For the peptidic ligands, a fluorescent dye was attached to the N-terminus of the peptide via an amino acid sequence KKGG (K=Lysine, G=Glycine), which provided solubility in addition to flexibility. In the case of peptides used for antibody target generation, fluorescein was replaced with carboxy biotin.

To cleave the peptides from the resin, a cocktail consisting of 1 mL TFA, 2.5% TSP (triisopropylsilane) and 2.5% water was used. The resin and cocktail were stirred at room temperature for approximately 3 to 4 hours. The resin beads were filtered off using glass wool, followed by rinsing with 2-3 mL of TFA. The peptide was precipitated with 40 mL of ice-cold ether and centrifuged at 3000-4000 rpm until the precipitate formed a pellet at the bottom of the centrifuge tube. The ether was decanted, and the pellet was resuspended in cold ether (40 mL) and centrifuged again; the process was repeated two to three times. During the final wash, 10 mL of purified water (such as that produced by Millipore's Analyzer Feed System) was added to 30 mL of cold ether, and the mixture was centrifuged again. The ether was decanted. The aqueous layer, containing the crude peptide, was transferred to a round bottom flask for lyophilization. Crude yields for peptide synthesis were usually approximately 90%. No unlabeled peptide was typically observed.

Cyclic peptides were generated by stirring the cysteine-containing crude peptides in an aqueous solution (1 mg/2-3 mL) with 20% DMSO overnight.

Peptides were purified by reverse phase semipreparative or preparative HPLC with a C4-silica column (Vydac, Hesperia, Calif.). The peptide chromatograms were monitored at 220 nm, which corresponds to the absorption of the amide chromophore. To ensure the presence of the fluorescein dye on the peptide, 495 nm was also observed. A solvent system of CH₃CN/TFA (acetonitrile/Trifluoroacetic acid; 100:0.01) and H₂O/TFA (water/Trifluoroacetic acid; 100:0.01) eluents at flow rates of 3 mL/min and 10 mL/min for semipreparative and preparative, respectively, were used. Dissolved crude peptides in purified water (such as that produced by Millipore's Analyzer Feed System) were injected at a scale of 1.5 mg and 5-10 mg peptide for semipreparative or preparative, respectively. The chromatogram shape was analyzed to ensure good resolution and peak shape. Gradient conditions for all peptides were typically 5 to 50% of CH₃CN/TFA (100:0.01) in 30 minutes. Purified peptide identity was confirmed by matrix-assisted laser desorption time-of-flight mass spectroscopy. Peptide cyclization typically resulted in both a change in the retention time by HPLC and a different mass by MALDI-TOF.

### EXAMPLE 5

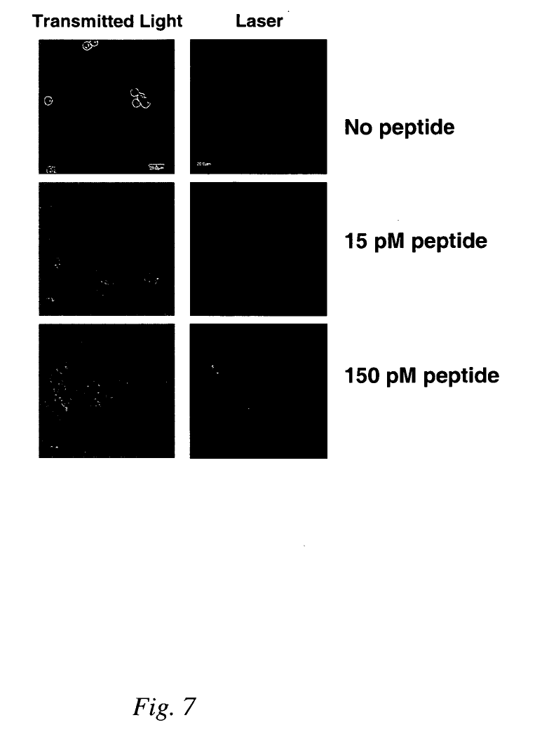

This Example illustrates screening of uPAR specific peptides in the cancer cell line RKO (ATCC CRL 2577). RKO cancer cells, which overexpress uPAR, were cultured in appropriate media in a 6 well plate to >80% confluence. Increasing concentrations (0-0.15 nM) of fluorescein-tagged peptide were added to live cells in full media and incubated for 6 hours. Following incubation, cells were removed from the wells with tryspin, washed three times with 1 mL phosphate-buffered saline and fixed using 1% glutaraldehyde. Cells were then mounted on slides for analysis with confocal microscopy. FIG. 7 is a micrograph at 80× magnification of RKO cancer cells following incubation with fluorescein tagged AESTYHHLSLGYMYTLN-NH2.

Cells receiving uPAR peptides at concentrations of at least 0.015 nM peptide had observable binding of peptides to all cells. Alternatively, one skilled in the art could utilize higher throughput optical analyzers (InCell 1000, Amersham Bioscience/GEHC) that can measure uptake of fluorescent molecules in 96 well plates.

### EXAMPLE 6

This Example illustrates the design and synthesis of peptides for targeting other biomarkers. An example of designing peptides that bind an integrin, αᵥβ₃ , is found in Wadih Arap, Renata Pasqualini, Erkki Ruoslahti, SCIENCE 279:377 (Jan. 16, 1998). In Arap et al., in vivo selection of peptide sequences using phage display libraries was used to isolate those that home specifically to tumor blood vessels. Two of these peptides, one containing an av integrin-binding Arg-Gly-Asp motif and the other an Asn-Gly-Arg motif, targeted αᵥβ₃ effectively in tumor vasculature.

Peptide sequences for use in targeting other biomarkers may be synthesized using the methods above.

### EXAMPLE 7

This Example illustrates advantages of the core/shell nanoparticle-based targeted MRI contrast agents over those of the prior art.

The analytical data for the core/shell nanoparticles, shown in Table 2, includes the hydrodynamic size, surface charge, the Si/Fe mass ratio for nanoparticles that contain Si, as well as the relaxivity values (R1, R2, and R2/R1) of the multiple core/shell particles described herein. Measurement of D_{H}, the surface potential (ζ), and the Si/Fe mass ratio (for samples with silane based coatings) are standard analyses performed to determine batch quality and purity.

TABLE 2
Analytical Data for 5 nm Coated SPIO Agents

| | | R1 | R2 | |
|---|---|---|---|---|
| Shell | D_{H} (nm) | (mM⁻¹ s⁻¹) | (mM⁻¹ s⁻¹) | R2/R1 |
| PEI-Silane | 13.8 ± 1.4 | 14.5 | 48.2 | 3.3 |

Aggregation. One analytical parameter for measuring nanoparticle aggregation is the hydrodynamic size as measured by dynamic light scattering (DLS) in aqueous solutions. For 5 nm SPIO PEI-Silane coated particles, a D_{H} value greater than about 30 nm is indicative of particle aggregation. Functionalization of 5 nm particles with PEI silane results in a coated nanoparticle with hydrated diameter of less than 15 nm and a dispersity of less than 10%. Further addition of targeting molecules to this coated particle will lead to an increase in size, including but not limited to up to 25-30 nm. In one embodiment, the final functionalized and targeted nanoparticle will have a diameter of less than 30 nm and a dispersity of ˜10%.

Relaxivity. Unfunctionalized 5 nm SPIO PEI-Silane coated particles have a R2/R1 ratio of 3.3. This value indicates a contrast agent with T1 and T2 properties and demonstrates increased relaxivity over particles described in the prior art.

Targeting. Using the available functionality on the coated nanoparticle, targeting molecules can be attached to specific markers of disease to target the particle to disease sites of interest. For example, targeting the nanoparticle to tumors overexpressing urokinase receptor (uPAR) could provide essential information regarding the biological activity and location of tumors following imaging. To accomplish this, targeting molecules would be attached to the coated nanoparticle by methods described above and retain their ability to specifically and tightly bind (Kd<1 mM) to their targets.

Blood clearance and biodistribution. A non-agglomerated, monodisperse targeted nanoparticle less than 30 nm in diameter will preferably have a blood half-life in humans of less than 12 hours but more than 1 hour. This may provide a maximal uptake at the site of interest (locus of disease) and decrease the background signal due to particles remaining in the vasculature. The physical characteristics of these targeted nanoparticles should allow for the particles to evade the RES and effectively target sites of interest. Smaller size (˜30 nm) and monodispersity should allow the particles to distribute in the body and not to traffic non-specifically to the liver and spleen before accumulating at disease loci.

Signal. Following administration of the targeted nanoparticle to the subject, imaging will be performed at an optimal timepoint hours after administration. In this manner, signal changes due to accumulation of the nanoparticles will be observed using optimized imaging protocols. In one example, imaging could be performed 24 hours after injection. At this timepoint, residual nanoparticles will no longer be found in the blood and targeted nanopaticles will be localized at a disease site (i.e. atherosclerotic lesion, tumor or other). Imaging using T2-specific pulse sequences will result in images where accumulated particles will result in net signal loss of more than 10% below that of background signal from surrounding tissue. This will provide needed clinical information.

### EXAMPLE 8

This Example illustrates the delivery of therapeutic agents by functionalized nanoparticles, wherein peptide-functionalized cationic nanoparticles deliver oligonucleotides to disease-specific sites for therapeutic purposes. In this example, cationic nanoparticles that have a functional shell such as polyethylene imine (PEI) may utilize available functional groups to covalently attach targeting molecules without completely neutralizing the cationic surface. Free oligonucleotides could then be added to the targeted cationic nanoparticles. The positive surface charge would allow for reversible binding of the negatively charged oligonucleotide. Upon forming this targeted complex, the complex may be administered to cells or to a mammalian subject. The targeted complex would locate the cell target of interest and, upon internalization of the complex, release the oligonucleotide for delivery to the cell.

### EXAMPLE 9

This Example illustrates the administration of the core/shell nanoparticle-based targeted MRI contrast agents to a subject in vivo. Animals were scanned by magnetic resonance imaging to generate “pre-injection” T2-weighted MR images of a rat anatomy. A specific region of interest (ROI) was the liver. Sterile core/shell nanoparticle-based targeted MRI contrast agents was then administered via tail vein injection to female Sprague-Dawley rats at a dose of 1 mg Fe/kg body weight or 5 mg Fe/kg body weight in a total injection volume of 600 microliters.

### EXAMPLE 10

This Example illustrates monitoring the core/shell nanoparticle-based targeted MRI contrast agents in vivo. Following initial administration of core/shell nanoparticle-based targeted MRI contrast agents, animals were transferred to cages for 24 hours and then imaged again to generate “post-injection” T2-weighted MR images of a rat anatomy. The liver was identified as a region of interest (ROI) and several images were obtained.

It will be understood that certain of the above-described structures, functions, and operations of the above-described embodiments are not necessary to practice the present invention and are included in the description simply for completeness of an exemplary embodiment or embodiments. In addition, it will be understood that specific structures, functions, and operations set forth in the above-described referenced patents and publications can be practiced in conjunction with the present invention, but they are not essential to its practice. It is therefore to be understood that the invention may be practiced otherwise than as specifically described without actually departing from the spirit and scope of the present invention as defined by the appended claims.

What is claimed is:

1. A targeted MRI contrast agent comprising:
a) an inorganic-based magnetic core;
b) an organic-based non-magnetic coating, comprising a silane, disposed about and bonded to the inorganic-based magnetic core such that, in the aggregate, the magnetic core and the non-magnetic coating provide for a core/shell nanoparticle; and
c) a targeting species attached to the core/shell nanoparticle such that, in the aggregate, the core/shell nanoparticle and the targeting species provide for a targeted MRI contrast agent.

2. The targeted MRI contrast agent of claim 1, wherein the inorganic-based magnetic core comprises a material selected from the group consisting of transition metals, alloys, metal oxides, metal nitrides, metal carbides, metal borides, and combinations thereof.

3. The targeted MRI contrast agent of claim 1, wherein the inorganic-based magnetic core comprises a material that is superparamagnetic.

4. The targeted MRI contrast agent of claim 3, wherein the inorganic-based magnetic core comprises iron oxide of the formula [Fe₂³⁺O₃]ₓ[Fe₃³⁺O₄]₁₋ₓ where 1≧x≧0.

5. The targeted MRI contrast agent of claim 4, wherein the inorganic-based magnetic core has a Mₛₐₜ value of at least about 60 emu/g Fe for a 5 nm inorganic-based magnetic core.

6. The targeted MRI contrast agent of claim 1, wherein the silane is selected from the group consisting of silane modified polyethylene imine, aminopropylsilane, 2-carboxyethylsilane, N-iodoacetyl aminopropylsilane, 3-isocyanatopropylsilane, 5,6-epoxyhexyltriethoxysilane, 3-isothiocyanatopropylsilane, and 3-azidopropylsilane.

7. The targeted MRI contrast agent of claim 1, wherein the organic-based non-magnetic coating is a polymer.

8. The targeted MRI contrast agent of claim 7, wherein the polymer comprises silane modified polyethylene imine.

9. The targeted MRI contrast agent of claim 1, wherein the organic-based non-magnetic coating is a non-polymer.

10. The targeted MRI contrast agent of claim 9, wherein the non-polymer is aminopropylsilane.

11. The targeted MRI contrast agent of claim 1, wherein the core/shell nanoparticle has a hydrodynamic diameter of less than about 100 nm.

12. The targeted MRI contrast agent of claim 1, wherein the core/shell nanoparticle has a hydrodynamic diameter of less than about 50 nm.

13. The targeted MRI contrast agent of claim 1, wherein the core/shell nanoparticle has a hydrodynamic diameter of less than about 30 nm.

14. The targeted MRI contrast agent of claim 1, wherein the targeting species is attached to the core/shell nanoparticle by a manner selected from the group consisting of via a covalent linkage, directly and via a linker species.

15. The targeted MRI contrast agent of claim 1, wherein the targeting species is selected from the group consisting of a peptide, a protein, an oligonucleotide; a small organic molecule, a peptide nucleic acid, and combinations thereof.

16. The targeted MRI contrast agent of claim 15, wherein the peptide is selected from the group consisting of AEPVYQYELDSYLRSYY (SEQ ID NO: 1), AEFFKLGPNGYVYLHSA (SEQ ID NO: 2), AELDLSTFYDIQYLLRT (SEQ ID NO: 3), AESTYHHLSLGYMYTLN (SEQ ID NO: 4), and combinations thereof.

17. The targeted MRI contrast agent of claim 1, wherein the targeted MRI contrast agent is made by a method comprising the steps of:
a) synthesizing a core of a nanoparticle;
b) synthesizing a shell of the nanoparticle so that the core of the nanoparticle is substantially covered by the shell; and
c) attaching a targeting molecule to the shell of the nanoparticle.

18. A method comprising the steps of:
a) providing a composition comprising:
i) an inorganic-based magnetic core;
ii) an organic-based non-magnetic coating, selected from the group consisting of silane modified polyethylene imine and aminopropylsilane, disposed about and bonded to the inorganic-based magnetic core such that, in the aggregate, the magnetic core and the non-magnetic coating provide for a core/shell nanoparticle; and
iii) a targeting species attached to the core/shell nanoparticle; and
b) using the composition as a contrast agent for MRI.

19. The method of claim 18, wherein the contrast agent is delivered to a cell in vitro.

20. The method of claim 19, wherein delivery of the contrast agent to the cell is monitored.

21. The method of claim 18, wherein the contrast agent is delivered to a subject in vivo.

22. The method of claim 21, wherein delivery of the contrast agent to the subject is monitored.

23. The method of claim 22, wherein monitoring delivery of the contrast agent is accomplished via an imaging technique selected from the group consisting of MRI, optical imaging, optical coherence tomography, computer tomography, positron emission tomography, and combinations thereof.
